(19) United States
(12) Patent Application Publication
(10) Pub. No.: US 2024/0085434 A1
(43) Pub. Date: Mar. 14, 2024

(54) Urinary Metabolites as Predictors of Acute Mountain Sickness Severity

(71) Applicant: Government of the United States as Represented by the Secretary of the Air Force, Wright-Patterson AFB, OH (US)

(72) Inventors: Camilla A. Mauzy, Enon, OH (US); James P. Karl, Roslindale, MA (US)

(73) Assignee: Government of the United States as Represented by the Secretary of the Air Force, Wright-Patterson AFB, OH (US)

(21) Appl. No.: 18/464,365

(22) Filed: Sep. 11, 2023

Related U.S. Application Data
(60) Provisional application No. 63/375,111, filed on Sep. 9, 2022.

(51) Int. Cl. G01N 33/68; G01N 24/08

(52) U.S. Cl. CPC G01N 33/6893; G01N 24/08; G01N 2800/50

(57) ABSTRACT

A method of predicting acute mountain sickness (AMS). The method includes collecting a urine sample from a subject and analyzing the urine sample for a quantity of at least one metabolite selected from the group consisting of creatine, taurine, N-methylhistidine, hypoxanthine, 1-methylnicotinamide, 4-hydroxyphenylpyruvate, acetylcarnitine, and 3-methylhistidine. The quantity is compared to a threshold value for the respective metabolite. Based on the comparison, it is determined whether the subject is susceptible to experience AMS at high altitudes.

Pursuant to 37 C.F.R. § 1.78(a)(4), this application claims the benefit of and priority to prior filed co-pending Provisional Application Ser. No. 63/375,111, filed Sep. 9, 2022, which is expressly incorporated herein by reference in its entirety.

### RIGHTS OF THE GOVERNMENT

The invention described herein may be manufactured and used by or for the Government of the United States for all governmental purposes without the payment of any royalty.

### FIELD OF THE INVENTION

The present invention relates generally to methods of predicting acute mountain sickness severity.

### BACKGROUND OF THE INVENTION

At high altitude, hypobaric hypoxia elicits a series of physiological responses that are highly variable in humans. While these responses assist in adapting to high altitude conditions, the response can also lead to development of acute mountain sickness (“AMS”) or life-threatening forms of altitude-induced illness, such as high-altitude cerebral edema (“HACE”) or high-altitude pulmonary edema (“HAPE”). Non-acclimatized, AMS-susceptible subjects usually develop AMS symptoms in 6 hrs to 12 hrs after a rapid ascent and exposure to high altitudes; symptoms generally resolving within 72 hrs of altitude exposure. While eventually self-resolving, severe AMS symptoms can be temporarily debilitating Such effects may be an unpleasant nuisance for leisure travelers but for military personnel, AMS can compromise occupational performance.

Prevention of AMS onset involves pharmaceutical and non-pharmaceutical approaches. Pharmaceutical prophylaxis has limitations as medications such as acetazolamide are associated with side effects that while mild may discourage use. Non-pharmaceutical approaches include pre-acclimatization by intermittent exposure to normobaric hypoxia or spending time at moderate altitude before ascending to higher elevations. While pre-acclimatization carries the benefit of reducing AMS, implementation can be logistically difficult.

However, without information on a prior history of AMS, it is difficult to identify which subjects would be at highest risk of severe AMS before ascent. There are no clinical or routine laboratory examinations that can be performed to determine AMS susceptibility. As such, there has been some interest in developing rapid molecular-based screening methods for that purpose. Once conventional approaches have been to develop a model for identifying subjects at risk of developing severe AMS and other forms of altitude-induced illness However, the model requires subjects to undergo an exercise test regimen, while breathing a hypoxic gas mixture, which is not amendable to widespread application. Moreover, the value and accuracy of such models have been questioned. Another conventional approach to evaluating predisposition has been to evaluate serum levels of ITIH4 347-35, ITIH1 205-214, and FGA 588-624) at sea level; however, the accuracy has not yet been established and the screening requires invasive blood collection. An ideal screening platform would be non-invasive (e.g., urine) and easy to implement.

Genetic factors have been regarded as key players in high-altitude adaptation, suggesting that genetic polymorphisms influence high altitude adaptation. It is possible that functional polymorphisms in key enzymes involved in physiologic pathways may drive occurrence and severity of AMS and that metabolite outputs yielded by these pathways can be determined using a metabolomics-based approach.

Metabolomics is a unique top-down approach that can be applied to study complex systems. Metabolite profiles are regarded as good indicators of an organism's physiology as such profiles measure an “end result” of multiple protein, gene, and environmental interactions. As such, applying metabolomic approaches to examine physiological alterations resulting from altitude adaptation may not only identify biomarkers for AMS susceptibility, but may also provide further insight into the physiologic pathways affecting AMS.

Thus, there remains a need for improved methods of identifying subjects having a predisposition to AMS. Furthermore, there is a great need for such methods to be non-invasive, amendable to widespread application, and easily implemented across a variety of environments.

### SUMMARY OF THE INVENTION

The present invention overcomes the foregoing problems and other shortcomings, drawbacks, and challenges of identifying subjects having a predisposition to AMS. While the invention will be described in connection with certain embodiments, it will be understood that the invention is not limited to these embodiments. To the contrary, this invention includes all alternatives, modifications, and equivalents as may be included within the spirit and scope of the present invention.

According to one embodiment of the present invention a method of predicting acute mountain sickness (AMS) is taught. The method includes collecting a urine sample from a subject and analyzing the urine sample for a quantity of at least one metabolite selected from the group consisting of creatine, taurine, N-methylhistidine, hypoxanthine, 1-methylnicotinamide, 4-hydroxyphenylpyruvate, acetylcarnitine, and 3-methylhistidine. The quantity is compared to a threshold value for the respective metabolite. Based on the comparison, it is determined whether the subject is susceptible to experience AMS at high altitudes.

Other embodiments of the present invention include a method evaluating acclimatization after exposure to high altitude and associated AMS. The method includes collecting a first urine sample from a subject at a first altitude of not more than 4900 ft (1500 m) above sea level and analyzing the first urine sample for a first quantity of at least one metabolite selected from the group consisting of creatine, taurine, N-methylhistidine, hypoxanthine, 1-methylnicotinamide, 4-hydroxyphenylpyruvate, acetylcarnitine, and 3-methylhistidine. The first quantity is compared to a first threshold value for the respective metabolite. The subject is then exposed to a second altitude that is greater than 4900 ft (1500 m) above sea level, a second urine sample from the subject is collected, and the second urine sample is analyzed for a second quantity of at least one metabolite selected from the group consisting of creatine, taurine, N-methylhistidine, hypoxanthine, 1-methylnicotinamide, 4-hydroxyphenylpyruvate, acetylcarnitine, and 3-methylhistidine. The second quantity is compared to a second threshold value for the respective metabolite, the first quantity, or both and, based on the comparison, it is determining whether the subject has acclimatized to the second altitude.

Additional objects, advantages, and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims.

### BRIEF DESCRIPTION OF THE DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIGS. 1A and 1B are a flow chart illustrating a method of using specific identified urinary metabolites as predictors to determine Acute Mountain Sickness (“AMS”) severity according to an embodiment of the present invention.

FIG. 2 is a schematic illustration a computing system suitable for comparative analysis according to embodiments of the present invention.

FIG. 3 graphically illustrates results of a principal component analysis score modeling urine data for AMS group at sea level and on day 1 of high altitude.

FIGS. 4A and 4B graphically illustrates an analysis of urinary creatine levels for AMS susceptible and NoAMS subjects, wherein FIG. 4A is data from ¹H NMR and FIG. 4B is data from a creatine enzyme-linked immunosorbent (“ELISA”) assay.

FIG. 5 is a graphical illustration of the study timeline described in the example.

FIG. 6 graphically illustrates results form an orthogonal projection onto latent structures-discriminant analysis modeling urinary metabolite data for AMS and No AMS at sea level.

FIGS. 7A-7D graphically illustrates a percent change in peak intensity between day 1 at high altitude and day 18 at high altitude relative to sea level for AMS susceptible subjects for creatine, N-methylhistidine, hypoxanthine, and acetylcarnitine, respectively, as generated by ¹H NMR Metabolomic analysis.

FIGS. 8A-8G graphically illustrates relative peak intensities ¹H NMR Metabolomic spectral data for samples collected from AMS susceptible and NoAMS resistant subjects at sea level, day 1 of high altitude, and day 18 of high altitude of taurine, N-methylhistidine, hypoxanthine, 1-methylnicotinamide, 4-hydroxyphenylpyruvate, acetylcarnitine, and 3-methylhistidine, respectively.

FIGS. 9A-9F graphically illustrate results of a correlation analysis between creatine and hypoxanthine for NoAMS subjects at sea level (FIG. 9A), day 1 at high altitude (FIG. 9B), and day 18 at high altitude (FIG. 9C), and for AMS susceptible subjects at sea level (FIG. 9D), day 1 at high altitude (FIG. 9E), and day 18 at high altitude (FIG. 9F).

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION OF THE INVENTION

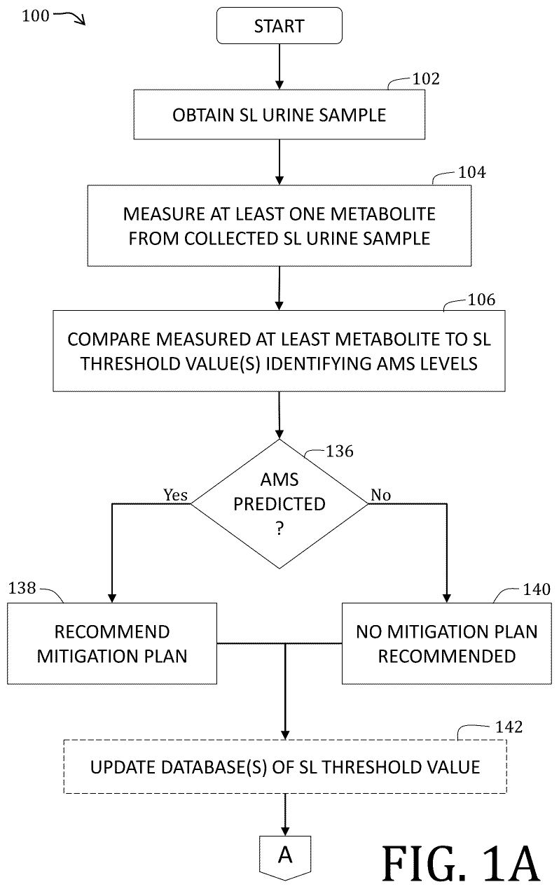

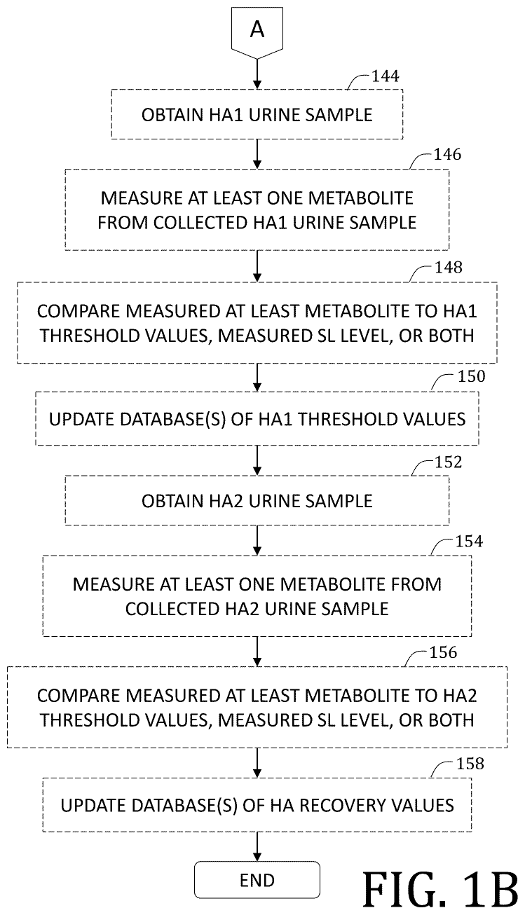

Referring now to the figures, and in particular to FIG. 1, a method 100 of using urinary metabolites as predictors of Acute Mountain Sickness (“AMS”) severity according to an embodiment of the present invention begins with obtaining a urine sample of a user at a first altitude. The first altitude may be sea level (“SL URINE SAMPLE,” Block 102) or an altitude of not more than 4900 ft (1500 m) above sea level. Hereinafter, sea level will be referenced as “SL.” Collection may be accomplished in accordance with conventional practices (clean catch, sterile urine bag, suprapubic aspiration, or urethral catheterization, for example) so long as a clean sample is obtained.

The SL urine sample is prepared in accordance with known procedures for metabolic analysis. Such preparation may include, but is not limited to, those know by skilled artisans in the preparation of samples for analytical analysis, such as by nuclear magnetic resonance (NMR), high performance liquid chromatography (HPLC), chemical assay, enzyme-linked immunosorbent assay (“ELISA”), and so forth. The samples may also be frozen for later analysis, if needed or required.

With the sample prepared, at least one metabolite in the SL urine sample is measured (Block 104) with the at least one metabolite selected from creatine, 4-hydroxyphenylpyruvate, taurine, N-methylhistidine, acetylcarnitine, hypoxanthine, 1-methylnicotinamide, and 3-methylhistidine. According to some embodiments, at least two metabolite of the SL urine sample is measured with the at least two metabolites selected from creatine, 4-hydroxyphenylpyruvate, taurine, N-methylhistidine, acetylcarnitine, hypoxanthine, 1-methylnicotinamide, and 3-methylhistidine. According to some embodiments, at least three metabolites of the SL urine sample is measured with the at least three metabolites selected from creatine, 4-hydroxyphenylpyruvate, taurine, N-methylhistidine, acetylcarnitine, hypoxanthine, 1-methylnicotinamide, and 3-methylhistidine. Yet other embodiments include at least four metabolite of the SL urine sample is measured with the at least four metabolites selected from creatine, 4-hydroxyphenylpyruvate, taurine, N-methylhistidine, acetylcarnitine, hypoxanthine, 1-methylnicotinamide, and 3-methylhistidine. Other embodiments have five or more metabolites of the SL urine sample measured with the five or more metabolites selected from creatine, 4-hydroxyphenylpyruvate, taurine, N-methylhistidine, acetylcarnitine, hypoxanthine, 3-methylhistidine, and 1-methylnicotinamide.

Measurement of metabolites may include nuclear magnetic resonance (NMR), such as ¹H NMR. Other analyses may include mass-spectroscopy (with or without HPLC, high performance liquid chromatography), gas chromatography, fluoroscopy, wet assay, or other methods of analysis that are known by those of ordinary skill in the art having the benefit of the disclosure made herein.

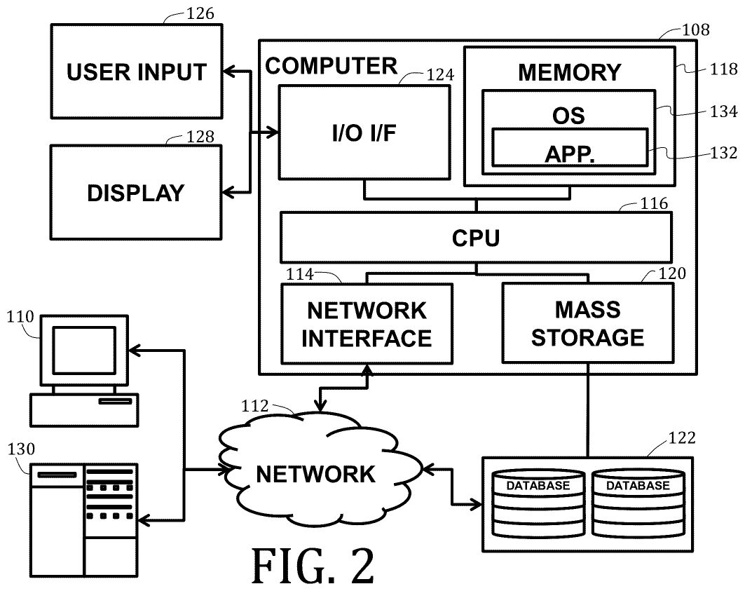

The measured metabolite may then be compared to a SL threshold value (Block 106). The comparison may be accomplished using a computer system 108, an exemplary system suitable for performing the method being illustrated in FIG. 2. The illustrative computing system 108 may be considered to represent any type of computer, computer system, computing system, server, disk array, or programmable device such as multi-user computers, single-user computers, handheld devices, networked devices, or embedded devices, etc. The computing system 108 may be implemented with one or more networked computers 110 using one or more networks 112, e.g., in a cluster or other distributed computing system through a network interface 114 (illustrated as “NETWORK I/F”). The computing system 108 will be referred to as “computer” for brevity's sake, although it should be appreciated that the term “computing system” may also include other suitable programmable electronic devices consistent with embodiments of the invention.

The computer 108 typically includes at least one processing unit 116 (illustrated as “CPU”) coupled to a memory 118 along with several different types of peripheral devices, e.g., a mass storage device 120 with one or more databases 122, an input/output interface 124 (illustrated as “I/O I/F”) coupled to a user input 126 and a display 128, and the Network I/F 114. The memory 118 may include dynamic random-access memory (“DRAM”), static random-access memory (“SRAM”), non-volatile random-access memory (“NVRAM”), persistent memory, flash memory, at least one hard disk drive, and/or another digital storage medium. The mass storage device 120 is typically at least one hard disk drive and may be located externally to the computer 108, such as in a separate enclosure or in one or more networked computers 110, one or more networked storage devices (including, for example, a tape or optical drive), and/or one or more other networked devices (including, for example, a server 130). The SL threshold value(s) may be stored in the memory 118 or the networked database 122, for example.

The CPU 116 may be, in various embodiments, a single-thread, multi-threaded, multi-core, and/or multi-element processing unit (not shown) as is well known in the art. In alternative embodiments, the computer 108 may include a plurality of processing units that may include single-thread processing units, multi-threaded processing units, multi-core processing units, multi-element processing units, and/or combinations thereof as is well known in the art. Similarly, the memory 118 may include one or more levels of data, instruction, and/or combination caches, with caches serving the subjects processing unit or multiple processing units (not shown) as is well known in the art.

The memory 118 of the computer 108 may include one or more applications 132 (illustrated as “APP.”), or other software program, which are configured to execute in combination with the Operating System 134 (illustrated as “OS”) and automatically perform tasks necessary for performing the method of FIGS. 1A and 1B, with or without accessing further information or data from the database(s) 122 of the mass storage device 120.

Those skilled in the art will recognize that the environment illustrated in FIG. 2 is not intended to limit the present invention. Indeed, those skilled in the art will recognize that other alternative hardware and/or software environments may be used without departing from the scope of the invention.

Referring again now to FIG. 1A, comparing the metabolite of the SL urine sample to the SL threshold value(s) leads to a determination as to whether AMS may be predicted (Decision Block 136). For instance, if the metabolite is taurine, N-methylhistidine, hypoxanthine, or 1-methylnicotinamide, AMS may be predicted if a level of the metabolite in the SL urine sample is less than the respective threshold value of metabolite. If the metabolite is creatine, 4-hydroxyphenylpyruvate, acetylcarnitine, or 3-methylhistidine, AMS may be predicted if the level of the metabolite in the SL urine sample is greater than the respective threshold value of the metabolite.

The skilled artisan may also appreciate that the multiples of the metabolite measured may improve confidence of the conclusion. For instance, if taurine and 4-hydroxyphenylpyruvate are both measured, AMS may be predicted if the level of taurine in the SL urine sample is less than the taurine threshold value and if the level of 4-hydroxyphenylpyruvate in the SL urine sample is greater than the 4-hydroxyphenylpyruvate threshold value. As such, multiple combinations two or more metabolites, three or more metabolites, four or metabolites, ore other combinations may be used and are included within the scope of various embodiments of the present invention.

With the comparison complete, and if AMS is predicted (“Yes” branch of Decision Block 136), then an AMS mitigation plan may be devised and implemented (Block 138). The AMS mitigation plan may include pharmaceutical and non-pharmaceutical approaches. If AMS is not predicted (“No” branch of Decision Block 136), then no AMS mitigation is recommended (Block 140); however, that is not to say that a mitigation plan should not be implemented to ease stress of HA exposure.

Optionally, the results of the measured at least one metabolite, whether AMS was predicted, whether AMS was experienced, or a combination thereof may be used to update the SL threshold values of the 118 (FIG. 2) or database 122 (FIG. 2) (Block 142)

Referring now to FIG. 1B, the method 100 may optionally continue after exposure to a second altitude, wherein the second altitude is generally greater than 4900 ft (1500 m) above sea level and hereafter referenced as “HA.” As such, and optionally, on a first day of HA exposure (“HA1”), a urine sample may be obtained in accordance with methods described above (Block 144). According to other embodiments the HA1 urine sample may be obtained within two days of HA exposure. In still yet other embodiments, the HA1 urine sample may be obtained within three days of HA exposures. For still yet other embodiments, a plurality of urine samples may be obtained in the first day, second day, third day, or combinations thereof after HA exposure. The HA1 urine sample may be prepared and analyzed in accordance with the methods described previously. In that regard, at least metabolite is measured from the HA1 urine sample (Block 146). This analysis may be similar to the analysis at SL; however, such is not required. Then at least one measured metabolite may then be compared to a HA1 threshold, which may be retrieved from memory 118 (FIG. 2) or database 122 (FIG. 2) (Block 148). If the comparison yields an AMS susceptible result (or that AMS is anticipate), and the subject is not yet exhibiting symptoms, then mitigation plans may be implemented (not show in FIG. 1B); otherwise, no mitigation may be necessary. Additionally, and optionally, the measured at least one metabolite and whether AMS was experienced may be used to update the HA1 threshold values of the memory 118 (FIG. 2) or database 122 (FIG. 2) to improve future analyses (Block 150). Additionally, the comparison may be used to indicate a level of acclimatization to the higher altitude wherein the measured at least one metabolite levels approximately return to pre-exposure levels.

Referring still to FIG. 1B, the method 100 may optionally include obtaining a urine sample some days after HA exposure and when acclimatization is expected (“HA2”) in accordance with methods described above (Block 152). The HA2 urine sample may be prepared and analyzed in accordance with the methods described previously. In that regard, at least metabolite is measured from the HA2 urine sample (Block 154). This analysis may be similar to the analysis at SL or HA1; however, such is not required. The at least one measured metabolite may then be compared to a HA2 threshold, which may be retrieved from memory 118 (FIG. 2) or database 122 (FIG. 2) (Block 156). The measured at least one metabolite and whether AMS was experienced may be used to update the HA2 threshold values of the memory 118 (FIG. 2) or database 122 (FIG. 2) to improve future analyses (Block 158), to indicate a level of acclimatization, or both.

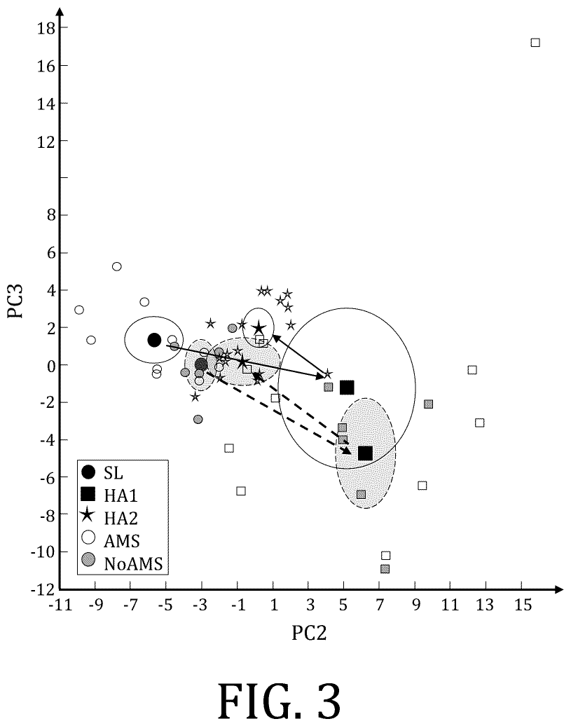

While not wishing to be bound by theory, principal component analysis results indicates that the urinary metabolite profiles for AMS and NoAMS groups changed significantly as the subjects moved from SL to HA and during a stay at HA, reflecting the subject's response to altitude environment. The changes in metabolite profiles from SL to HA reflect alterations in metabolic pathways, which are likely driven by complex adaptive changes in multiple biological systems responding to hypobaric hypoxia. The AMS group displayed greater variation in data at HA1 (FIG. 3) compared to the NoAMS group, highlighting AMS subject's diverse responses to high altitude conditions. The observations that metabolite profiles for both groups were distinct at SL and became more similar at high altitude after tie and acclimatization, suggest the existence of the urinary metabolite signatures for AMS susceptibility that may be apparent even before exposure to altitude-induced stress. The eight urinary metabolites that drove the separation of AMS from NoAMS subjects at sea level are identified.

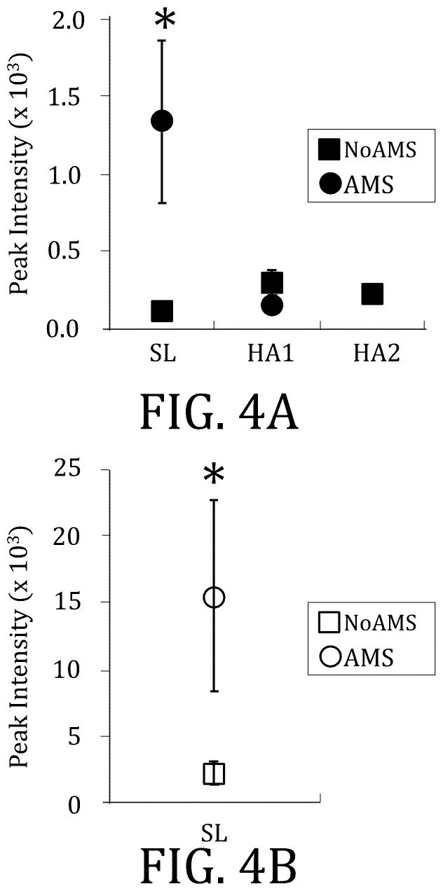

Of the metabolite alterations seen at SL, creatine had the highest contribution to the PCA segregation of NoAMS subjects. The average urinary creatine level in AMS susceptible subjects was 12-fold greater at sea level than NoAMS subjects (FIGS. 4A and 4B). The difference could result from one or more factors including: (1) a higher dietary intake of creatine-containing foods, (2) a lower conversion rate of creatine to phosphocreatine and creatinine, and (3) decreased cellular retention of creatine. Dietary protein intake did not differ between groups at SL and volunteers reported compliance with instructions not to consume any supplements. While urinary phosphocreatine excretion was not examined, urine creatinine levels did not differ between groups. Normalization using creatine to standardize against urine volume did not affect the predictive nature of the metabolite set. The simplest and most likely explanation for the higher creatine excretion rate in AMS subjects is decreased cellular retention.

Lower creatine cellular retention at sea level would lead to an increased rate of urinary elimination, limiting cellular availability of the substrate required for phosphocreatine synthesis once shifted to hypoxic conditions. The implication is that in AMS susceptible subjects, cells may have an existing deficiency in an energy supply needed to cope with altitude-induced hypoxia in the low oxygen environment. Hypoxia is known to affect cellular ATP production through downregulation of several tricarboxylic cycle enzymes as well as compromising electron transport chain complexes. Thus, increased urinary excretion of creatine at SL in AMS susceptible subjects may suggest that existing deficiencies of cellular creatine levels may increase hypoxia sensitivity.

Hypoxanthine was also among the metabolites that classified AMS and NoAMS groups at SL. Hypoxanthine is a naturally occurring purine degradation by-product, and cellular levels are associated with cellular levels of creatine. For example, hypoxanthine supplementation has been shown to reverse hypoxia-induced depletion of cellular creatine and phosphocreatine pools. Cellular levels of hypoxanthine may be lower in AMS subjects which could, in turn, impair the cellular retention of creatine and account for its higher urinary excretion.

Hypoxanthine, a metabolite that classified AMS and NoAMS groups at SL is a naturally occurring purine degradation by-product, and cellular levels are associated with cellular levels of creatine. For example, hypoxanthine supplementation has been shown to reverse hypoxia-induced depletion of cellular creatine and phosphocreatine pools. Findings of the present study suggest that cellular levels of hypoxanthine may be lower in AMS subjects which could, in turn, impair the cellular retention of creatine and account for its higher urinary excretion.

AMS susceptible subjects also demonstrated significantly lower taurine excretion at sea level and Day 1 at altitude relative to NoAMS subjects. Previous studies have suggested that this biogenic amine plays a significant role in protecting cells against hypoxia-induced damage. Further, under hypoxic conditions, taurine supplementation has been shown to improve cardiovascular function in pigs, attenuate vascular remodeling in rats, and prevent learning impairment and increase survival time in mice. Although, taurine's mechanisms of protection against hypoxia-mediated decrements are not well understood, taurine may act as a potent endogenous agent to induce cellular growth despite oxygen deficiency and improve both osmotic status and calcium homeostasis. The lower urinary excretion of taurine seen at SL and Day 1 at altitude in AMS subjects may reflect an increase in degradation of this metabolite.

Acetylcarnitine plays a critical role in cellular energy metabolism and has been shown to play a role in cellular responses to hypoxia-induced stress. Some studies have demonstrated that daily supplementation of acetylcarnitine to rats during hypoxic exposure ameliorated hypoxia-induced deficits in spatial working memory, oxidative stress, and apoptotic cascades, suggesting that this metabolite plays a significant role in the body's response to hypoxic stress. In the current study, urinary acetylcarnitine excretion in AMS susceptible subjects was higher than for NoAMS subjects at SL. This may suggest that the cellular stores of this metabolite were lower in AMS subjects, and their increased susceptibility to AMS may be mediated by alteration in energy or lipid metabolism.

Urinary N-methylhistidine is formed in the body through methylation of peptide-bound histidine in muscle actin and myosin and eliminated in urine after protein breakdown. Urinary excretion of N-methylhistidine is regarded as useful indicator for muscle protein breakdown provided that the subject has a meat-free diet. Though dietary protein can affect urinary excretion, it is unlikely that the diet was driving the lower N-methylhistidine in AMS susceptible vs. NoAMS subjects as dietary protein intake did not differ between the groups at SL. Of note, previous studies have shown that the levels of N-methylhistidine are altered in subjects sensitive to high altitude. For example, plasma levels of methylhistidine have previously been shown to increase in subjects with HAPE compared to controls.

Increased urinary excretion at sea level of 4-hydroxyphenylpyruvate (“4-HPPA”) in AMS subjects suggest a pre-existing alteration in the phenylalanine catabolism pathway, the 4-HPPA degradation pathway, or both may contribute to AMS susceptibility. However, phenylalanine and tyrosine levels in the urine were not statistically different between groups. As the downstream of 4-HPPA degradation pathway was not investigated, a more thorough study examining the molecular mechanisms for excessive 4-HPPA urinary elimination is being examined in current evaluations.

### Example

The following example illustrates particular properties and advantages of some of the embodiments of the present invention. Furthermore, this is an example of reduction to practice of the present invention and confirmation that the principles described in the present invention are therefore valid but should not be construed as in any way limiting the scope of the invention.

The analyses used archived samples and data from a study designed to assess the efficacy of a higher protein diet for preserving fat-free mass during high altitude (“HA”; 4,300 m) sojourn from C. E. BERRYMAN et al., “Severe negative energy balance during 21 d at high altitude decreases fat-free mass regardless of dietary protein intake: a randomized controlled trial,” FASEB J., Vol. 32 (2018) 894-905, the disclosure of which is incorporated herein by reference, in its entirety.

The study was approved by the Institutional Review Board at the United States Army Research Institute of Environmental Medicine (USARJEM) in Natick, MA and was registered on https://clinicaltrials. gov/, NCT02731066. The protocol is incorporated herein by reference, in its entirety.

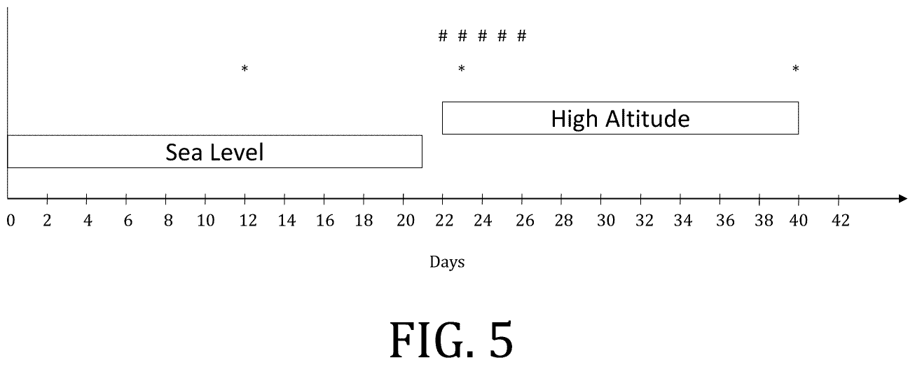

Seventeen healthy, unacclimatized, physically active men (aged 18-42 years) participated in the study. Although, study enrollment was open to both sexes, no women volunteered to participate. The human study was a randomized, controlled trial consisting of two phases conducted over 43 consecutive days. During the 21-day first phase (a diagram of the study is provided in FIG. 5), participants resided at sea level (“SL”), consumed a self-selected weight maintaining diet, maintained habitual exercise routines, and were free living but visited the laboratory daily. On day 21, participants were flown from Boston, MA to Denver, CO and then were placed on supplemental oxygen until arriving at the summit of Pike's Peak, CO (4,300 m) the following morning (day O at HA). Participants then resided for the next 21 days at the United States Army Research Institute of Environmental Medicine Maher Memorial Laboratory, Pike's Peak, CO (phase 2; HA). During HA, participants were under constant supervision, consumed a controlled and measured diet, and engaged in prescribed physical activity. Participants started consuming controlled diets on day 1, the first full day of residence at 4,300 m, and continued until the end of the sojourn at HA (phase 2). Diets contained either a standard amount of protein (1.1 g/kg/day; n=8) or higher amount of protein (2.1 g/kg/day; n=9), and were designed to induce weight loss, which is common during military training and operations, and during HA sojourn.

The prevalence and severity of AMS was assessed using the shortened version of the Environmental Symptoms Questionnaire (“ESQ”). Days of testing are indicated with “#” in FIG. 5. The ESQ was administered twice daily during the first 5 days at HA and used to calculate AMS weighted cerebral factor scores. Peak scores were recorded from all participants during the first 48 hrs at HA and were used to categorize AMS severity as mild (˜0.7 and <1.53), moderate (˜1.53 and <2.63), and severe (˜2.63). The analyses described below used two group identifiers: AMS and NoAMS. Subjects who scored less than 1.53 were designated as belonging to the NoAMS (no/mild AMS symptoms) group (n=6), while those with scores greater than 1.53 were designated as the AMS (moderate/severe AMS symptoms) group (n=11).

Urine samples (indicated with “*” in FIG. 5) obtained at sea level 9 days prior to accent to altitude (SL) and at high altitude on days 1 (HA1) and 18 (HA2) were used for the analyses described herein. Collections began at 0730 on SL day 12 and 0700 on HA days 1 and 18 following an overnight fast and required participants to collect all urine produced over 2 hrs. During that 2-hr period, participants consumed their individualized standard or higher protein breakfast on all 3 test days. Day 12 was the only day participants ate their diet group-specific breakfast during SL. Aliquots of the SL samples were frozen ¹H nuclear magnetic resonance (“NMR”) metabolomic analyses and shipped to Wright Patterson Air Force Base (WPAFB), Dayton, OH for analyses. Upon arrival at WPAFB, all urine samples were stored at −80° C. The preparation of urine samples for ‘H NMR spectral data acquisition followed the procedure described in Sibomana et al. (2017).

All proton NMR spectra were acquired using a Varian INOVA NMR instrument operating at 600 MHz and a probe temperature of 25° C. NMR spectral data acquisition and processing are routinely performed in our laboratory. These procedures were conducted as known to those skilled in the art.

Multivariate data analyses were conducted on binned and scaled spectral data. Binned NMR data were scaled to the entire dataset chosen as reference by subtracting each bin value from the mean value for the corresponding bin in the reference data (whole dataset), then dividing this value by the SD of the reference data (auto-scaling).

FIG. 3 illustrates results of a Principal Component Analysis (“PCA”) used as an unsupervised analysis technique and provided a first approach for data visualization. The PCA model was constructed based on the data for AMS group at SL and HA1. Data for the AMS group at HA2 and NoAMS group at SL, HA1, and HA2 were then superimposed onto the PCA scores plot. The quality of data clustering in this PCA model was evaluated using Davies-Bouldin (D. L. DAVIES and D. W. BOULDIN, “A cluster separation measure,” IEEE Trans. Pattern Anal. Mach. Intell. PAMI-1 (1979), 224-227) and Silhouette (P. ROUSSEEUW, “Silhouettes: a graphical aid to the interpretation and validation of cluster analysis,” J. Comput. Appl. Math., Vol. 20 (1987) 53-65) indexes. The Davies-Boulclin index (“DBI”) is defined as a ratio between the within group distances (intra group scatter) and the between group distances (group's separation). The lower the DBI value, the better the cluster separation and the tightness inside the groups. The Silhouette index is a measure of how similar a data point is to its own cluster (group) compared to other clusters (groups): the higher the value, the better the data point matches to its own duster (group) and the poorer it matches to neighboring clusters (groups).

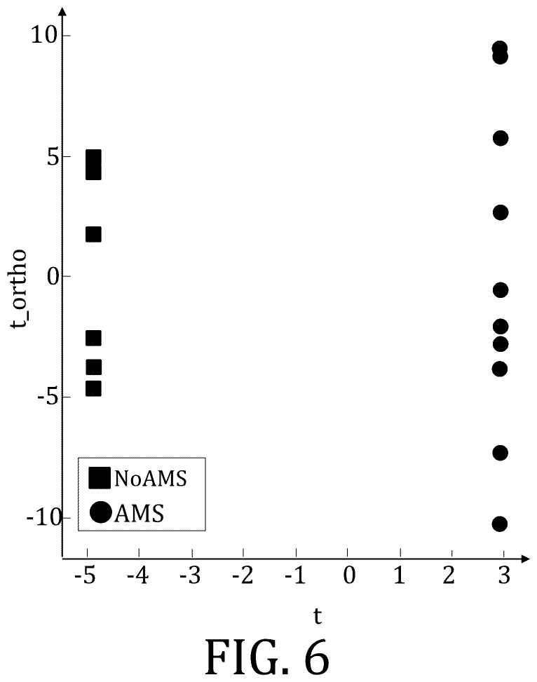

FIG. 6 illustrates the results of an Orthogonal Projection onto Latent Structures-Discriminant Analysis (“OPLS-DA”) used as a supervised technique to classify data and identify salient features that allow class separation of AMS vs. NoAMS at SL, HA1, and HA2. The Q² (coefficient of prediction) metric was used to evaluate the predictive ability of the OPLS models. A permutation test was also performed to evaluate the significance of the Q² metric. This test involved repeatedly permuting the data labels and re-running the discrimination analysis, resulting in a distribution of the Q² scores. The Q² from the correctly labeled data is then compared to the distribution to determine the significance of the model at a specified alpha (set herein as a=0.01). A receiver operator characteristic (“ROC”) curve was also used as a secondary validation of an OPLS binary model and the area under the curve (“AUC”) was calculated. Evaluation of the significance of this AUC value was conducted using the same permutation procedure as described above.

The variable selection (salient bins) from OPLS-DA was statistically evaluated by comparing bin loading, commonly referred to as coefficients, to calculated null distributions in order to select for significance. The null distribution for each bin was determined by refitting the OPLS model to datasets, in which each bin was independently and randomly permuted to remove any correlation between it and AMS/NoAMS groups. The true OPLS model loading was then compared to the resulting null distribution of loadings, and values in the tail (greater than 99.5% or less than 0.5% of the null distribution; corresponding to a=0.01) were assumed to contribute significantly to the model. The permutation was initially repeated 1,000 times for each bin and those near significant loadings (greater than 92.5% or less than 7.5% of the null distribution; corresponding to a=0.2) were selected for 500 additional permutations (total 1,500).

Normalized NMR spectra (PQN method; see above) were used to quantify metabolite resonances determined to be important for group classification. Quantification of specific metabolite resonances was accomplished using an interactive spectral deconvolution algorithm in MATLAB. The deconvolution tool fits a defined spectral region using a combination of tunable baseline shapes (spline, v-shaped, linear, or constant) and a Gauss-Lorentz peak-fitting function. Metabolite peak intensities (total peak area) represent a semi-quantitative assessment of urine metabolites since this biofluid accumulates in the bladder over a variable period of time (i.e., 8 hrs) and its volume cannot be controlled. Although, the PQN method of spectral normalization helps to adjust for variable urine concentrations, absolute quantitative amounts of each metabolite are not reported. However, the semiquantitative metabolite measurements reported herein do allow a relative comparison between samples.

Nuclear magnetic resonance spectral regions identified as significant by OPLS-DA were compared between time points (SL, HA1, and HA2) and AMS vs. NoAMS, and specific resonances were assigned to metabolites with the aid of literature, on-line databases (HMDB, http://www.hmdb.ca/, www.bmrb.wisc.edu, etc.), and by “spiking” samples with known compounds, if necessary. Signal intensities were integrated to obtain relative measures of metabolite concentrations at each time point.

Creatine assays were performed on additional archived urine samples collected at the same time as samples used for the NMR analysis. Assays were conducted using an Abeam (Cambridge, United Kingdom) creatine activity assay kit (ab65339) according to manufacturer instructions.

A repeated measures MANOVA was conducted to examine effects of time and AMS status (AMS vs. NoAMS) on urine metabolite profiles. Only metabolites identified in OPLS-DA as significant were subjected to MANOVA. For metabolites demonstrating time-by-AMS group interactions (p<0.05), Levene's and Welch's tests were conducted to assess the equality of variances between the data for SL, HA1, and HA2 or AMS vs. NoAMS groups for each metabolite using statistical software package JMP® 11.0.0 (SAS Institute, Cary, NC, United States). If Levene's test was significant (p≤0.05), then a Welch's nonparametric ANOVA test was used to determine if there were significant differences in the mean values between groups for the metabolite of interest. If the Levene's test was not significant, significance was tested using a one-way ANOVA (t-test). If both Levene's and Welch's tests were significant (p≤0.05), a pairwise Welch test was performed for all pairs of groups. No false discovery rate correction was applied to the data since OPLS-DA and MANOVA were used to down-select metabolites. Only metabolites identified by both data analysis methods were considered as statistically significant. Results are expressed as mean±SEM and are considered statistically significant at p s 0.05. Cohen's d (effect size) was used as a measure of the magnitude of changes in the level of each urinary metabolite noted at HA1 and HA2 relative to SL by subtracting the value obtained for SL from those obtained for HA (HA1 or HA2) and assessing the difference relative to the pooled SDs for HA (HA1 or HA2) and SL.

The mean peak AMS-weighted cerebral factor score for AMS subjects (2.25±0.18; n=11) was significantly elevated (p<0.05) compared to in NoAMS subjects (0.78±0.18; n=6). AMS severity (i.e., NoAMS vs. AMS) was unrelated to diet group. PCA analysis indicated that the urinary metabolite profiles for both groups changed over the time course of the study with the AMS group displaying greater variation in data at HA1 compared to NoAMS, as shown in FIG. 2. PCA clearly separated urinary profiles for AMS from NoAMS at all time points, with differences being most apparent at SL. The urinary profiles for both groups at HA2 indicated a trajectory returning toward SL. Mapping positions for NoAMS at SL and at HA2 partially overlapped, indicating some similarities in metabolite profiles. In contrast, there is a clear separation in profiles within the AMS group at these two time points.

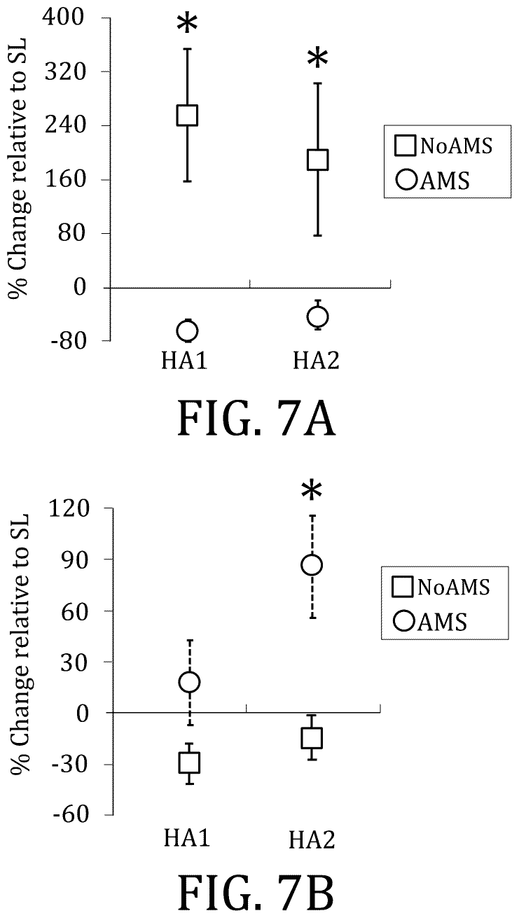

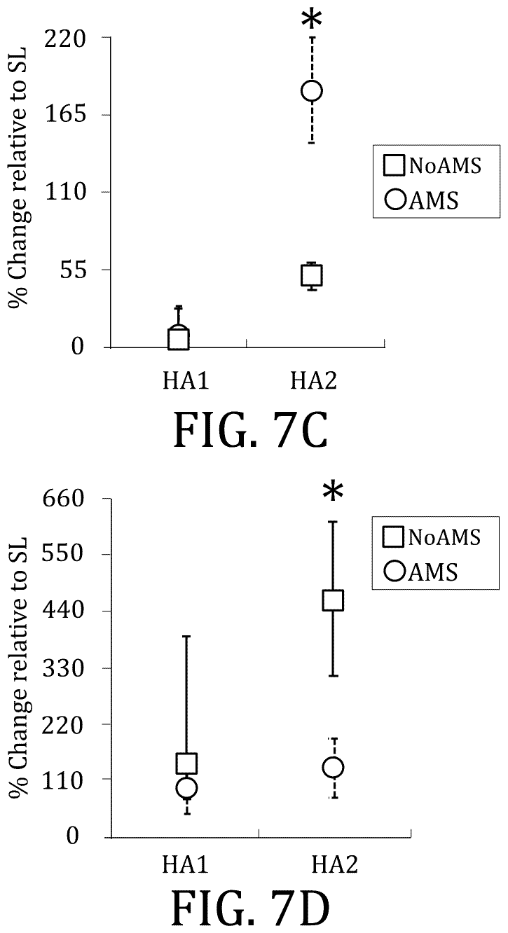

Changes in urinary metabolite levels that occurred from SL to HAI and SL to HA2 indicated that changes in the levels of only four metabolites differed between AMS or No AMS groups (FIGS. 7A-7D). The metabolites included creatine (energy metabolism), two amino acid derivatives consisting of N-methylhistidine and acetylcarnitine, and hypoxanthine (nucleotide derivative). FIGS. 7A-7D illustrate a percent change in peak intensity of creatine (FIG. 7A), N-methylhistidine (FIG. 7B), hypoxanthine (FIG. 7C), and acetylcarnitine (FIG. 7D) at HA1 and HA2 relative to SL for urine samples collected from AMS susceptible (data indicated with dots) and NoAMS resistant subjects (data indicated with squares). Asterisk denotes significant difference between groups (p≤0.05).

As shown in FIG. 7A, urinary creatine levels decreased 64% from SL to HA1 in the AMS group but increased by 256% in the NoAMS group. At HA2, creatine levels were 42% lower than that noted at SL in the AMS group but increased by 190% from SL to HA2 in the NoAMS group. The levels of hypoxanthine (FIG. 7C) and acetylcarnitine (FIG. 3D) increased by 182% and 135%, respectively, from SL to HA1 for the AMS group, while in the NoAMS group, levels increased by 51% and 463%, respectively. At HA2, N-methylhistidine levels (FIG. 7B) for AMS increased by 86% relative to SL, while NoAMS subjects decreased by 15%. It is noteworthy to indicate that creatine, hypoxanthine, and N-methylhistidine were among the metabolite classifiers of these two groups at SL.

Orthogonal projection onto latent structures-discriminant analysis comparing AMS and NoAMS at SL (FIG. 6) yielded a Q² value of LO (p=0.001), a predictive accuracy of 100% (leave-1-out cross validation) and an AUC value of LO (p=0.001). Further, the T-score scatter plots of the data confirmed that the urine metabolite profiles for the AMS group clustered together and were separated from the NoAMS group. Examination of metabolites that classified these two groups at SL indicated that creatine was the strongest driver of separation between the two groups (FIGS. 4A and 4B). Spectra from the ¹H NMR analysis showed that AMS subjects had (p≤0.05) higher relative peak intensity for creatine at SL (1.34±0.52) as compared to NoAMS subjects (0.11±0.03; FIG. 4A). These observations were confirmed by a secondary method using creatine assay analyses (FIG. 4B).

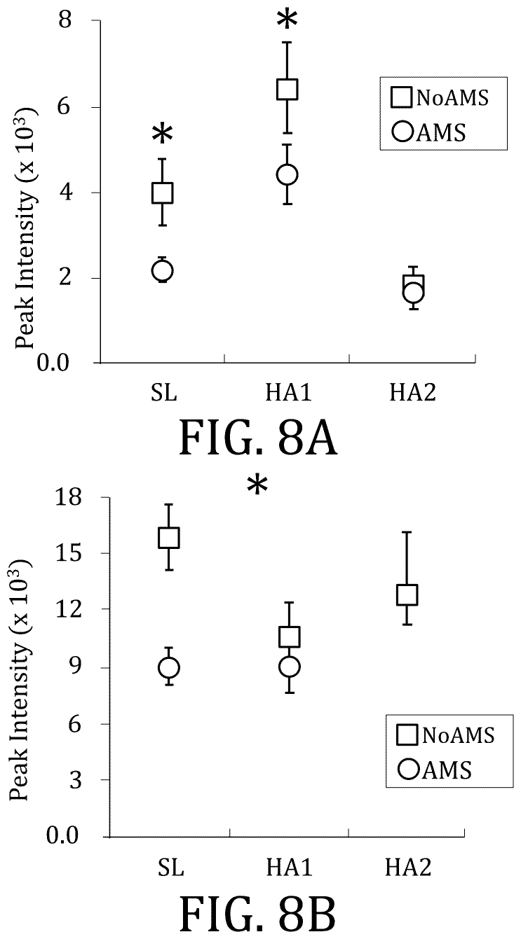

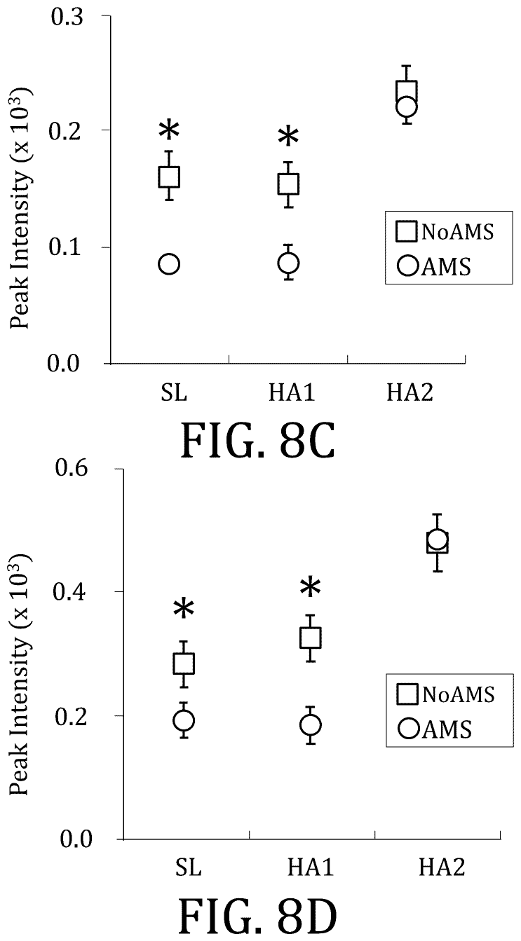

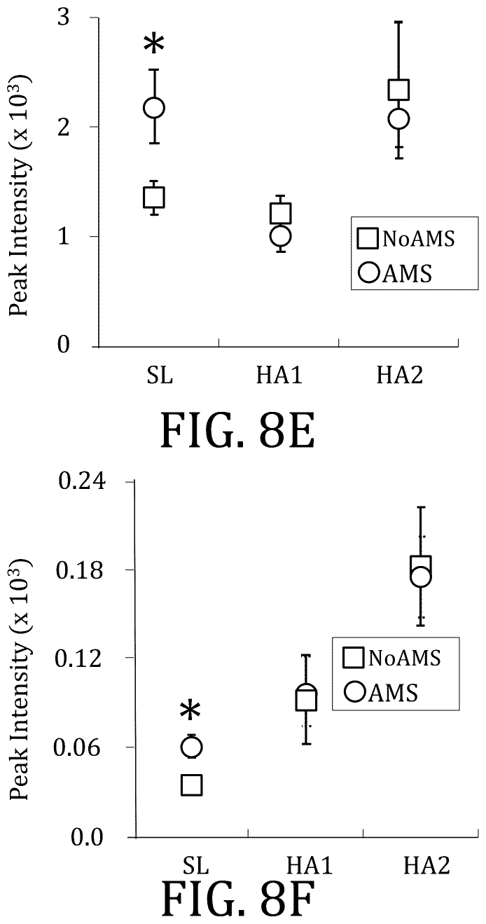

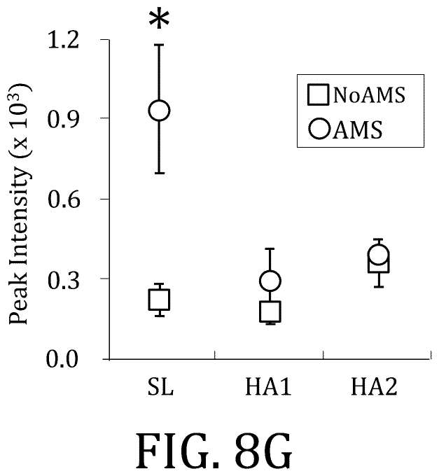

Additional metabolites driving discrimination between groups at SL included 4-hydroxyphenylpyruvate, taurine, N-methylhistidine, acetylcarnitine, hypoxanthine, and two unidentified metabolites (FIGS. 8A-8G). Urinary excretion levels of taurine (FIG. 8A), N-methylhistidine (FIG. 8B), hypoxanthine (FIG. 8C), and 1-methylnicotinamide (FIG. 8D) were lower in AMS vs. NoAMS at SL, while the levels of 4-hydroxyphenylpyruvate (FIG. 8E), acetylcarnitine (FIG. 8F), 3-methylhistidine (FIG. 8G) were elevated in the AMS group. The levels of taurine, hypoxanthine, and 1-methylnicotinamide for AMS subjects were still lower than values obtained for the NoAMS group at HA1, but these differences between groups disappeared at HA2.

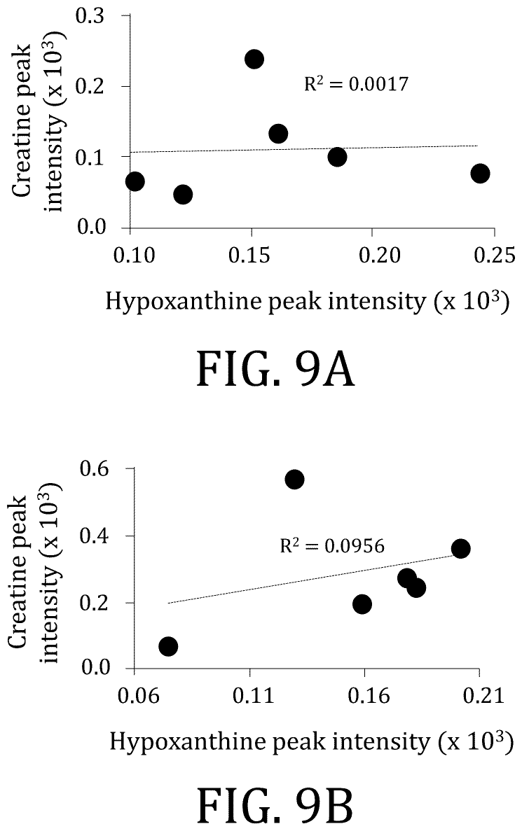

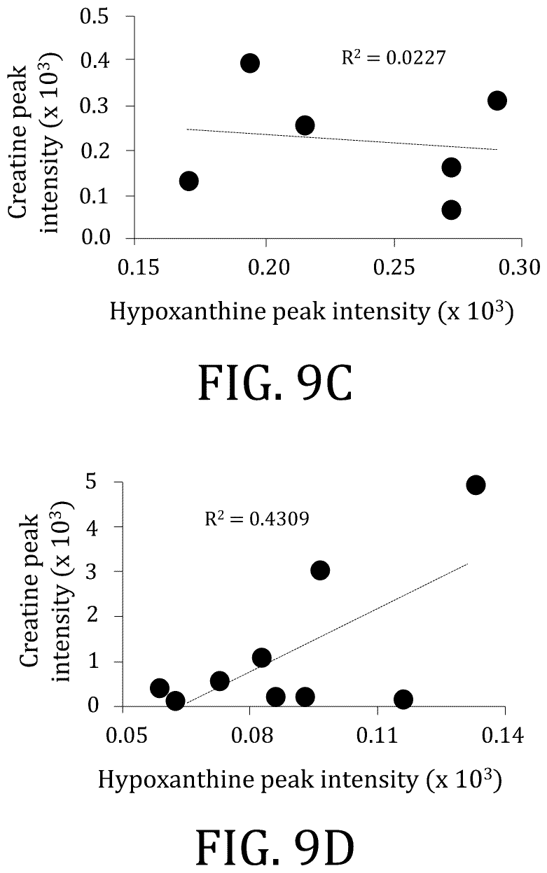

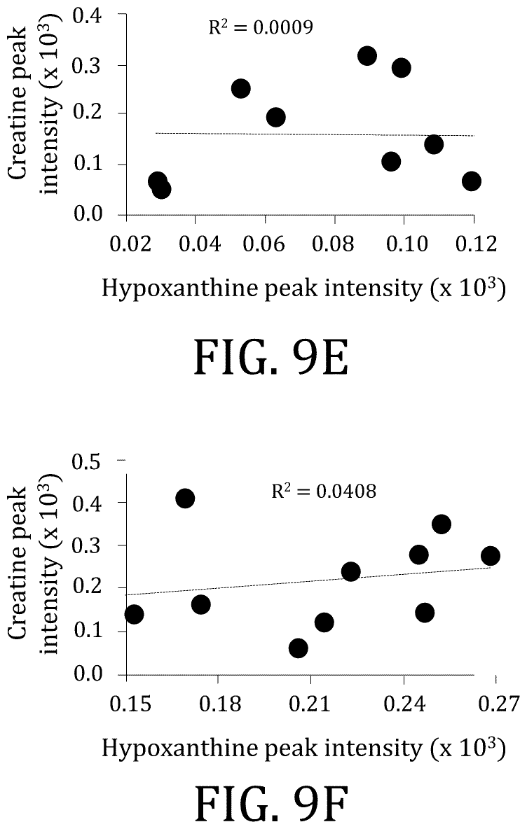

FIGS. 9A-9F illustrate the results form a correlation analysis between urinary levels of creatine and hypoxanthine for NoAMS subjects at SL (FIG. 9A), HA1 (FIG. 9B), and HA2 (FIG. 9C), and AMS-susceptible subjects at SL (FIG. 9D), HA1 (FIG. 9E), and HA2 (FIG. 9F).

While the present invention has been illustrated by a description of one or more embodiments thereof and while these embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the scope of the general inventive concept.

What is claimed is:

1. A method of predicting acute mountain sickness (AMS), the method comprising:
collecting a urine sample from a subject;
analyzing the urine sample for a quantity of at least one metabolite selected from the group consisting of creatine, taurine, N-methylhistidine, hypoxanthine, 1-methylnicotinamide, 4-hydroxyphenylpyruvate, acetylcarnitine, and 3-methylhistidine;
comparing the quantity to a respective threshold value; and
based on the comparison, determining whether the subject is susceptible to experience AMS at high altitudes.

2. The method of claim 1, wherein the determination further comprises:
indicating susceptibility when the quantity of taurine, N-N-methylhistidine, hypoxanthine, or 1-methylnicotinamide is below the respective threshold value.

3. The method of claim 1, wherein the determination further comprises:
indicating susceptibility when the quantity of creatine, 4-hydroxyphenylpyruvate, acetylcarnitine, or 3-methylhistidine is above the respective threshold value.

4. The method of claim 1 wherein the determination further comprises:
indicating susceptibility when the quantity of at least one of taurine, N-N-methylhistidine, hypoxanthine, or 1-methylnicotinamide is below a first threshold value; and
indicating susceptibility when the quantity of at least one of creatine, 4-hydroxyphenylpyruvate, acetylcarnitine, or 3-methylhistidine is above a second threshold value.

5. The method of claim 1, wherein analyzing the urine sample includes a metabolomic analysis or targeted quantitation or semi-quantitative method.

6. The method of claim 5, wherein the metabolomic analysis or targeted quantitation method is selected from the group consisting of an Nuclear Magnetic Resonance (NMR) spectra, liquid chromatography with mass spectroscopy (LC/MS), high-performance liquid chromatography with mass spectroscopy (HPLC/MS), liquid chromatography with tandem mass spectroscopy (LC/MS-MS), gas chromatography with mass spectroscopy (GC/MS), quantitation by capture element in enzyme-linked immunosorbent assay (ELISA), lateral flow assay (LFA), or biochemical assay.

7. The method of claim 6, wherein quantitation by capture element in ELISA includes evaluation of an antibody, a nanobody, or a peptide.

8. The method of claim 1, wherein analyzing the urine sample includes a lab method or a point-of-care method.

9. The method of claim 1, wherein the urine is collected at sea level.

10. The method of claim 1, wherein urine is collected at an altitude of not more than 4900 ft (1500 m) above sea level.

11. The method of claim 1, further comprising:
normalization of the collected urine sample using creatine to standardize against urine sample volume.

12. A method of evaluating acclimatization after exposure to high altitude and associated acute mountain sickness (AMS), the method comprising:
collecting a first urine sample from a subject at a first altitude of not more than 4900 ft (1500 m) above sea level;
analyzing the first urine sample for a first quantity of at least one metabolite selected from the group consisting of creatine, taurine, N-methylhistidine, hypoxanthine, 1-methylnicotinamide, 4-hydroxyphenylpyruvate, acetylcarnitine, and 3-methylhistidine;
comparing the first quantity to a respective first threshold value;
exposing the subject to a second altitude greater than 4900 ft (1500 m) above sea level;
collecting a second urine sample from the subject at the second altitude;
analyzing the second urine sample for a second quantity of at least one metabolite selected from the group consisting of creatine, taurine, N-methylhistidine, hypoxanthine, 1-methylnicotinamide, 4-hydroxyphenylpyruvate, acetylcarnitine, and 3-methylhistidine;
comparing the second quantity to a respective second threshold value, the first quantity, or both; and
based on the comparison, determining whether the subject has acclimatized to the second altitude.

13. The method of claim 12, wherein analyzing the urine sample includes a metabolomic analysis or targeted quantitation or semi-quantitative method.

14. The method of claim 13, wherein the metabolomic analysis or targeted quantitation method is selected from the group consisting of an Nuclear Magnetic Resonance (NMR) spectra, liquid chromatography with mass spectroscopy (LC/MS), high-performance liquid chromatography with mass spectroscopy (HPLC/MS), liquid chromatography with tandem mass spectroscopy (LC/MS-MS), gas chromatography with mass spectroscopy (GC/MS), quantitation by capture element in enzyme-linked immunosorbent assay (ELISA), lateral flow assay (LFA), or biochemical assay.

15. The method of claim 14, wherein quantitation by capture element in ELISA includes evaluation of an antibody, a nanobody, or a peptide.

16. The method of claim 13, further comprising:
normalization of the first and second urine samples using creatine to standardize against urine sample volume.
